(19) United States
(12) Patent Application Publication
(10) Pub. No.: US 2021/0079339 A1
(43) Pub. Date: Mar. 18, 2021

(54) FERMENTATION PRODUCTION PROCESS OF PAECILOMYCES HEPIALIS CS-4

(71) Applicant: JIANGXI GUOYAO PHARMACEUTICAI, LLC, NANCHANG (CN)

(72) Inventors: BIAO WU, NANCHANG (CN); YIBIN WAN, NANCHANG (CN); YOUQUN GE, NANCHANG (CN); FEIHONG ZUO, NANCHANG (CN); MING YANG, NANCHANG (CN); LIN HE, NANCHANG (CN); JINJIN LI, NANCHANG (CN)

(21) Appl. No.: 17/105,438

(22) Filed: Nov. 25, 2020

Related U.S. Application Data
(63) Continuation of application No. PCT/CN2018/119032, filed on Dec. 4, 2018.

(30) Foreign Application Priority Data
May 25, 2018 (CN) 201810545731.2

(51) Int. Cl. C12N 1/14; C12M 1/16

(52) U.S. Cl. CPC C12N 1/14; C12M 21/16

(57) ABSTRACT

A fermentation production process of paecilomyces hepialis Cs-4 including steps of inoculating Cs-4 strains on a slant culture medium, inoculating Cs-4 strains into a first culture medium in a shake flask, inoculating Cs-4 strains into a second culture medium in a seed tank, inoculating Cs-4 strains into a third culture medium in a propagation tank, and inoculating Cs-4 strains into a fourth culture medium in a fermentation tank. Fermentation broth obtained after fermentation is used as Cs-4 strains to be cultivated in the first culture medium in the shake flask. The slant culture medium includes histidine. The first culture medium in the shake flask includes the histidine and vitamin B₁. The second culture medium in the seed tank includes purine. The third culture medium in the propagation tank includes the purine and pyrimidine. The fourth culture medium in the fermentation tank includes purines and the pyrimidine.

### TECHNICAL FIELD

The present disclosure relates to a field of microorganisms technology, and in particular to a fermentation production process of paecilomyces hepialis Cs-4.

### BACKGROUND

Cordyceps sinensis is only one of more than 190 kinds of cordyceps in China. It is mainly produced on the Qinghai-Tibet Plateau and is also referred to as “cordyceps”. Cordyceps is a complex of larvae of the ergot family fungus Cordyceps parasitizing on the larvae of Helicoptera insects and the corpse of the larvae. Main active ingredients of the cordyceps comprises cordycepin, cordycepic acid, cordyceps polysaccharide and other substances that are beneficial to human body absorption. Cordyceps sinensis is a traditional medicinal tonic in China. It is natured and sweet, and has functions of tonifying lung and kidney, relieving coughs, benefiting deficiencies, and nourishing essence. According to clinical studies, Cordyceps sinensis has effects of nourishing lung yin, tonifying kidney yang, relieving cough and resolving phlegm, anti-cancer and anti-ageing, and is a product to rehabilitate yin and yang. Cordyceps sinensis is configured for pulmonary tuberculosis hemoptysis, impotence, spermatorrhea, etc. Cordyceps sinensis is precious and scarce due to its irreplaceable pharmacological and pharmacological effects and harsh ecological environment. With continuous improvement of human living standard, people's demand for Cordyceps sinensis is increasing while the resource of Cordyceps sinensis is decreasing, making the contradiction between supply and demand more and more prominent. In order to solve the problem, in recent years, Chinese biomedical researchers have isolated several strains of Cordyceps fungus and attempted to produce Cordyceps mycelium to replace natural Cordyceps sinensis using an artificial fermentation method. Through unremitting efforts, several companies have produced products that are similar to natural Cordyceps sinensis in terms of pharmacology, toxicology, and efficacy composition, and are able to realize large-scale production under modern conditions.

paecilomyces hepialis Cs-4 is a strain isolated from fresh Cordyceps collected in Qinghai. The strain was identified in the Institute of Microbiology, Chinese Academy of Sciences. Fermented product of paecilomyces hepialis Cs-4 comprises similar components to natural Cordyceps, such as adenine, nucleosides, urine. pyrimidine, mannitol, ergosterol and other effective ingredients. Further, pharmacological effect of the paecilomyces hepialis Cs-4 is better than natural Cordyceps and the toxicity is less than nature Cordyceps. Therefore, large-scale and high-quality industrial fermentation production of paecilomyces hepialis Cs-4 is of great significance for promoting people's health and improving quality of life.

Fermentation and cultivation of Cordyceps strains is a prerequisite for preparation of Cordyceps products. Conventional fermentation processes of the Cordyceps strains have problems such as low fermentation yield, long fermentation time and high costs. Therefore, fermentation processes such as static fermentation, batch fermentation and fed-batch fermentation are developed, but these fermentation processes still have various problems, such as high bacterial infection rate, long fermentation cycle, high cost, and low content of the active ingredients in fermentation products. Therefore, it is necessary to provide a fermentation process with high yield, low cost, and high content of the active ingredients in the fermented cordyceps powder products.

### SUMMARY

An object of the present disclosure is to provide a fermentation production process of paecilomyces hepialis Cs-4, which improves a yield of the fermentation product and content of active ingredients, shortens fermentation time, and reduces costs.

To be specific, in view of deficiencies in the prior art, the present disclosure provides following technical solutions:

A fermentation production process of paecilomyces hepialis Cs-4, comprises following steps:

step 1: inoculating Cs-4 strains on a slant culture medium and cultivating the Cs-4 strains for 7-9 days at a constant temperature in a range of 15−20° C.; wherein the slant culture medium comprises histidine;

step 2. inoculating the Cs-4 strains cultivated in step 1 into a first culture medium in a shake flask under aseptic conditions; placing the shake flask on a shaking table and cultivating the Cs-4 strains in the shake flask under light conditions for 7-9 days at a constant temperature in a range of 15-20° C.; wherein the first culture medium in the shake flask comprises the histidine and vitamin B₁;

step 3. inoculating the Cs-4 strains cultivated in step 2 in an amount ranging from 3-5% by weight into a second culture medium in a seed tank under aseptic conditions; wherein the second culture medium in the seed tank comprises purine;

step 4. inoculating the Cs-4 strains cultivated in step 3 in an amount ranging from 9-12% by weight into a third culture medium in a propagation tank under aseptic conditions; wherein the third culture medium in the propagation tank comprises purine and pyrimidine;

step 5. inoculating the Cs-4 strains cultivated in step 4 in an amount ranging from 5-20% by weight into a fourth culture medium in a fermentation tank and obtaining fermentation broth after cultivation in the fermentation tank is finished; wherein the fourth culture medium in the fermentation tank comprises purine and the pyrimidine;

or, the Cs-4 strains cultivated in step 5 are obtained by:

inoculating the fermentation broth obtained in step 5 in an amount ranging from 1-2% by weight into the first culture medium in the shake flask, and then cultivating the fermentation broth under conditions of step S2, and then repeating steps 3-5 to ferment and cultivate the Cs-4 strains

The purine and the pyrimidine are added to liquid fermentation culture mediums, which significantly increases content of nucleosides in cordyceps fungus powder prepared by the fermentation production process.

Optionally, the purine is selected from one or two of adenine and guanine. The pyrimidine is uracil.

Optionally, based on weight-to-volume ratio (g/mL), the slant culture medium comprises glucose in an amount ranging from 2-4%, peptone in an amount ranging from 0.2-0.4%, bran in an amount ranging from 0.4-0.8%, potassium dihydrogen phosphate in an amount ranging from 0.1-0.3%, magnesium sulfate in an amount ranging from 0.03-0.05%, the histidine in an amount ranging from 0.1-0.3%, agar in an amount ranging from 2-4%, and water provide for balance. The pH of the slant culture medium is nature. The first culture medium in the shake flask comprise the glucose in an amount ranging from 1-5%, the peptone in an amount ranging from 0.2-0.4%, the bran in an amount ranging from 0.4-0.6%, the potassium dihydrogen phosphate in an amount ranging from 0.1-0.3%, the magnesium sulfate in an amount ranging from 0.03-0.05%, zinc sulfate in an amount ranging from 0.03-0.05%, the histidine in an amount ranging from 0.1-0.3%, the vitamin B₁ in an amount ranging from 0.05-0.1%, and the water provided for balance. The pH of the first culture medium in the shake flask is nature.

Optionally, the second culture medium in the seed tank, the third culture medium in the propagation tank, and the fourth culture medium in the fermentation tank comprise notoginseng powder. After adding notoginseng powder, the cordyceps fungus powder obtained by the fermentation production process has effects of notoginsenoside and ginsenoside as well as the original effect of cordyceps. In addition, after adding the purine and the notoginseng powder to the fermentation culture medium, content of ergosterol is significantly increased.

Optionally, based on weight-to-volume ratio (g/mL), the second culture medium in the seed tank comprises hot-pressed soybean meal powder in an mount ranging from 2-4%, glucose in an mount ranging from 2-4%, sucrose in an mount ranging from 2-4%, potassium dihydrogen phosphate in an mount ranging from 0.2-0.4%, magnesium sulfate in an mount ranging from 0.05-0.1%, zinc sulfate in an mount ranging from 0.05-0.1%, the histidine in an mount ranging from 0.1-0.2%, the vitamin B₁ in an mount ranging from 0.02-0.3%, soybean oil in an mount ranging from 0.1-0.2%, adenine in an mount ranging from 0.05-0.2%, notoginseng powder in an mount ranging from 0.4-0.6%, and water provided balance. The pH of the second culture medium in the seed tank is nature.

Optionally, based on weight-to-volume ratio (g/mL), the third culture medium in the propagation tank comprises hot-pressed soybean meal in an mount ranging from 2-4%, glucose in an mount ranging from 2-4%, sucrose in an mount ranging from 2-4%, potassium dihydrogen phosphate in an mount ranging from 0.1-0.2%, magnesium sulfate in an mount ranging from 0.04-0.06%, zinc sulfate in an mount ranging from 0.03-0.05%, the histidine in an mount ranging from 0.1-0.2%, the vitamin B₁ in an mount ranging from 0.1-0.2%, soybean oil in an mount ranging from 0.2-0.3%, adenine in an mount ranging from 0.03-0.1%, guanine in an mount ranging from 0.03-0.08%, uracil in an mount ranging from 0.05-0.2%, notoginseng powder in an mount ranging from 0.5-0.7%, and water provided for balance. The pH of the third culture medium in the propagation tank is nature.

Optionally, based on weight-to-volume ratio (g/mL), the fourth culture medium in the fermentation tank comprises hot-pressed soybean meal in an mount ranging from 3-4%, glucose in an mount ranging from 1-2%, sucrose in an mount ranging from 1-2%, potassium dihydrogen phosphate in an mount ranging from 0.2-0.3%, magnesium sulfate in an mount ranging from 0.04-0.06%, zinc sulfate in an mount ranging from 0.04-0.06%, the histidine in an mount ranging from 0.2-0.3%, the vitamin B₁ in an mount ranging from 0.2-0.3%, soybean oil in an mount ranging from 0.2-0.3%, adenine in an mount ranging from 0.05-0.12%, guanine in an mount ranging from 0.03-0.08%, uracil in an mount ranging from 0.05-0.2%, notoginseng powder in an mount ranging from 0.5-1.0%, and water provided for balance. The pH of the fourth culture medium in the fermentation tank is nature.

Optionally, the shaking table in step 2 is a reciprocating shaking table. A shaking frequency of the shaking table is in a range of 140±5 times/min. Light used in the light condition is red light. The Cs-4 strains in the seed tank are cultivated at a constant temperature in a range of 16±3° C. Pressure of the seed tank is in a range of 0.02-0.05 MPa. Ventilation volume of the seed tank is 1:0.5-1.8 v/v·min. The Cs-4 strains in the seed tank are statically cultivated for 4-5 days.

Optionally, the Cs-4 strains in the propagation tank are cultivated at a constant temperature in a range of 16±3° C. Pressure of the propagation tank is in a range of 0.02-0.05 MPa. Ventilation volume of the propagation tank is 1:0.15-0.4 v/v·min. The Cs-4 strains in the propagation tank are statically cultivated for 5-6 days.

Optionally, the Cs-4 strains in the fermentation tank are cultivated at a constant temperature in a range of 16±3° C. Pressure of the fermentation tank is in a range of 0.02-0.05 MPa. Ventilation volume of the fermentation tank is 1:0.65-0.9 v/v·min. The Cs-4 strains in the fermentation tank are stirred and cultivated for 4-5 days at a rotating speed ranging from 40-60 rpm.

Optionally, ventilation volume of the fermentation tank is 1:0.75 v/v·min.

Compared with the prior art, the Cs-4 strains obtained after cultivated in the shake flask grow vigorously and produce many spores, providing excellent Cs-4 strains for cultivating in the seed tank. Components of the fermentation culture mediums of various levels is also slightly different, which ensures nutrient supply for the growth of the Cs-4 strains at different stages, and the obtained Cs-4 strains has high yield and high nutritional value. The seed tank and the propagation tank do not need to be stirred for cultivation, which saves costs.

The culture medium comprises amino acids, the purine and the pyrimidine. The Cs-4 strains cultivated by fermentation production process grow vigorously and the yield is high, and the content of nucleosides in the prepared cordyceps fungus powder is significantly increased. After adding the notoginseng powder to the culture mediums, the notoginsenoside is detected from the cordyceps fungus powder and the content of ergosterol is significantly increased. The present disclosure also finds that the purine and the pyrimidine are able to promote a utilization of the notoginseng powder by the Cs-4 strains, and increase the content of the ginsenoside and the notoginsenoside in the cordyceps fungus powder.

After cultivation of the Cs-4 strains in the fermentation tank is completed, part of the fermentation broth is inoculated into the first culture medium in the shake flask as seeds and the Cs-4 strains are cultivated in the shake flask again, and then the Cs-4 strains are cultivated in the seed tank, the propagation tank, and the fermentation tank sequentially. eliminating the need for cultivation on the slant culture medium. Thus, the fermentation process cycle is shorted and the costs are reduced. In addition, the fermentation broth is used as seeds and is cultivated in the shake flask and cultivation scales are enlarged step-by-step, and the cordyceps fungus powder obtained by the fermentation production process has a high content of various active ingredients. Further, the whole fermentation process has a high degree of automation, simple operation and is suitable for industrial production.

### DETAILED DESCRIPTION

A fermentation production process of paecilomyces hepialis Cs-4 of the present disclosure, comprise steps of cultivating the Cs-4 strains on a slant culture medium, cultivating Cs-4 strains in a first culture medium of a shake flask; cultivating Cs-4 strains in a second culture medium of a seed tank, cultivating the Cs-4 strains in a third culture medium of a propagation tank, and cultivating the Cs-4 strains in a fourth culture medium of a fermentation tank After cultivating the Cs-4 strains in the fermentation tank, fermentation broth is obtained. The fermentation broth obtained is inoculated into the shake flask in an amount ranging from 1-2% by weight. The remaining fermentation broth is filtered and dried to prepare Cs-4 cordyceps fungus powder. The 1-2% fermentation broth is equal to the Cs-4 strains obtained on the slant culture medium and is cultivated in the shake flask, and the remaining steps are repeated, such that scales of the Cs-4 strains are increasing step-by-step to obtain the fermentation broth again, and then a portion of the fermentation broth is inoculated into the culture medium in the first culture medium in the shaking flask again. Then, the fermentation process is repeated again. Generally, at a first fermentation production process, the Cs-4 trains cultivated on the slant culture medium is used as the Cs-4 strains being cultivated in the shake flask. After the fermentation broth is obtained, a portion of the fermentation broth is used as the Cs-4 strains to be cultivated in the shake flask for at least 10 times to ferment to produce Cs-4 mycelium, and then prepare the cordyceps fungus powder.

In one embodiment, the second culture medium in the seed tank comprises adenine ranging from 0.05-0.2% by weight and notoginseng powder ranging from 0.4-0.6% by weight. The third culture medium in the propagation tank comprises the adenine ranging from 0.03-0.1% by weight, guanine ranging 0.03-0.08% by weight, uracil 0.05-0.2%, and the notoginseng powder ranging from 0.5-0.7% by weight. The fourth culture medium in the fermentation tank comprises the adenine ranging from 0.05-0.12% by weight, the guanine ranging from 0.03-0.08% by weight, uracil ranging from 0.05-0.2% by weight, and the notoginseng powder ranging from 0.5-1.0% by weight. The pH of each culture medium is natural, generally in a range of 5.0-6.5. The Cs-4 strains in the shake flask are cultivated under irradiation of red light, and a wavelength of the red light ranges from 622-760 nm.

A preparation method of the slant culture medium and the first culture medium in the shake flask culture medium is as follows:

weighing bran filtered by a 40-mesh sieve according to a predetermined amount;

calculating an amount of water added according to 6 g bran per 1000 mL of water;

adding the water and heating mixture to boiling and boiling for 30 minutes;

filtering the mixture to obtain filtrate; and

adding other components into the filtrate; heating and/or adding water to dissolve, adding water to a constant volume, separating and sterilizing it.

A preparation method of the second culture medium in the seed tank is as follows: measuring 50% by weight of the water, adding components of the second culture medium to the water and heating to dissolve, then adding water to a constant volume, and sterilizing it. A preparation method of the third culture medium in the propagation tank and a preparation method of the fourth culture medium in the fermentation tank are same as the preparation method of the second culture medium in the seed tank except that components of the third culture medium in the propagation tank and components of the fourth culture medium in the fermentation tank are different with components of the second culture medium in the seed tank.

A sterilization method of various culture mediums is high-pressure steam sterilization with a steam pressure in a range of 0.09-0.12 Mpa, a temperature in a range of 121±1° C., and a sterilization time in a range of 30-40 minutes.

The present disclosure will be explained with specific embodiments below.

It should be noted that materials and equipment involved in the method of the present disclosure are all commercially available. Among them, the paecilomyces hepialis Cs-4 strain was deposited in the General Microbiology Center of China Microbial Culture Collection Management Committee on Oct. 21, 1997. The deposit number is CGMCC NO. 0327. The Cs-4 strain is isolated from fresh Cordyceps in Qinghai and can also be obtained commercially.

### Embodiment 1

The slant culture medium comprises 2% of glucose, 0.2% of peptone, 0.4% of bran, 0.1% of potassium dihydrogen phosphate, 0.03% of magnesium sulfate, 1% of histidine, 2% of agar, and water provide for balance. The pH of the slant culture medium is nature. Then, an inoculation spatula is applied to pick up the Cs-4 strains of 1 cm² that has been stored, and the Cs-4 strains are evenly spread on the slant culture medium and cultivated for 7 days at a temperature of 15° C. and a humidity of 85%. When the Cs-4 strains grow to maturity, an appearance inspection is performed. The cultivated Cs-4 strains on the slant culture medium has follow characteristics: spores and hyphae are plump, white or light yellow fluffy. Slant culture medium with poorly-growing Cs-4 strains are removed, and the slant culture medium with well-grown Cs-4 strains are stored in a refrigerator at 1-8° C. for later use.

The first culture medium in the shake flask comprise 1% of the glucose, 0.2% of the peptone, 0.4% of the bran, 0.1% of the potassium dihydrogen phosphate, 0.03% of the magnesium sulfate, 0.03% of zinc sulfate, 0.1% of the histidine, 0.05% of the vitamin B₁, and the water provided for balance. The pH of the first culture medium in the shake flask is nature.

Under aseptic conditions, the inoculation spatula is configured to take 1 cm² of the vigorously growing Cs-4 strains on the slant culture medium into the first culture medium in the shake flask. The shake flask is placed on a shaking table and the Cs-4 strains are shaken and cultivated at 15° C. for 7 days. A shaking frequency of the shaking table is 135 times/min. During an entire cultivation process in the shake flask, red light with a wavelength of 622-760 nm is used for irradiation. After the cultivation process in the shake flask is completed, the Cs-4 strains cultivated in each shake flask are collected into an inoculation steel cylinder.

The second culture medium in the seed tank comprises 2% of hot-pressed soybean meal powder, 2% of the glucose, 2% of sucrose; 0.2% of the potassium dihydrogen phosphate, 0.05% of the magnesium sulfate, 0.05% of the zinc sulfate 0.1% the histidine, 0.02% of the vitamin B₁, 0.1% of the soybean oil, 0.05% of the adenine, and water provided for balance. The pH of the second culture medium in the seed tank is nature. Under aseptic conditions, 3% by weight of the Cs-4 strains cultivated in the shake flask is inoculated into the second culture medium in the seed tank by pressure-difference method. The Cs-4 strains in the seed tank are cultivated at a constant temperature of 16° C. Pressure of the seed tank is 0.02 MPa, and ventilation volume of the seed tank is 1:0.5 v/v·min. The Cs-4 strains in the seed tank are statically cultivated for 4 days.

The third culture medium in the propagation tank comprises 2% of hot-pressed soybean meal powder, 2% of the glucose, 2% of sucrose; 0.1% of the potassium dihydrogen phosphate, 0.04% of the magnesium sulfate, 0.03% of the zinc sulfate; 0.1% the histidine, 0.02% of the vitamin B₁, 0.2% of the soybean oil, 0.05% of the adenine, 0.05% of uracil, and water provided for balance. The pH of the third culture medium in the propagation tank is nature. Under aseptic conditions, 9% by weight of the Cs-4 strains cultivated in the seed tank is inoculated into the third culture medium in the propagation tank by the pressure-difference method. The Cs-4 strains in the propagation tank are cultivated at a constant temperature of 16° C. Pressure of the propagation tank is 0.02 MPa, and ventilation volume of the seed tank is 1:0.15 v/v·min. The Cs-4 strains in the propagation tank are statically cultivated for 5 days.

The fourth culture medium in the fermentation tank comprises 3% of hot-pressed soybean meal powder, 1% of the glucose, 1% of sucrose; 0.2% of the potassium dihydrogen phosphate, 0.04% of the magnesium sulfate, 0.04% of the zinc sulfate; 0.2% the histidine, 0.2% of the vitamin B₁, 0.2% of the soybean oil, 0.05% of the adenine, 0.05% of uracil, and water provided for balance. Under aseptic conditions, 5% by weight of the Cs-4 strains cultivated in the propagation tank is inoculated into the fourth culture medium in the fermentation tank by the pressure-difference method. The strains in the fermentation tank are cultivated at a constant temperature of 16° C. Pressure of the fermentation tank is 0.02 MPa, and ventilation volume of the fermentation tank is 1:0.75 v/v·min. The Cs-4 strains in the fermentation tank are stirred and cultivated for 4 days at a rotating speed of 40 rpm. After cultivation in the fermentation tank is started, samples of the fermentation broth are taken every 24 hours to determine the pH, reducing sugars, and amino nitrogen. When pH<7, the reducing sugar<1.5%, the amino nitrogen<0.4 mg/mL, and bacteria concentration≥14%, the cultivation in the fermentation tank ends. Then the fermentation tank is opened to separate the fermentation liquid from solid and liquid, and the solid is dried and crushed to obtain Cs-4 cordyceps fungus powder.

Compared with the cordyceps powder prepared without adding purine and pyrimidine to liquid fermentation culture medium, content of nucleosides in the Cs-4 cordyceps fungus powder obtained in the embodiment is significantly increased, and content of ergosterol is also increased.

### Embodiment 2

The difference between embodiment 2 and embodiment 1 is: the second culture medium in the seed tank comprises 0.4% of the notoginseng powder, the third culture medium in the propagation tank comprises 0.5% of the notoginseng powder, and the fourth culture medium in the fermentation tank comprise 5% of the notoginseng powder. The Cs-4 strains are fermented and cultivated according to the same process as in Embodiment 1, and finally Cs-4 cordyceps fungus powder is obtained. Compared with the cordyceps fungus powder obtained of Embodiment 1, the content of ergosterol in the Cs-4 cordyceps fungus powder prepared in this embodiment is significantly increased, and the Cs-4 cordyceps fungus powder in the embodiment comprises notoginsenoside.

### Embodiment 3

The slant culture medium comprises 3% of glucose, 0.3% of peptone, 0.6% of bran, 0.2% of potassium dihydrogen phosphate, 0.04% of magnesium sulfate, 0.2% of histidine, 3% of agar, and water provide for balance.

The first culture medium in the shake flask comprise 3% of the glucose, 0.3% of the peptone, 0.5% of the bran, 0.2% of the potassium dihydrogen phosphate, 0.04% of the magnesium sulfate, 0.04% of zinc sulfate, 0.2% of the histidine, 0.07% of the vitamin B₁, and the water provided for balance.

The second culture medium in the seed tank comprises 3% of hot-pressed soybean meal powder, 3% of the glucose, 3% of sucrose; 0.3% of the potassium dihydrogen phosphate, 0.08% of the magnesium sulfate, 0.06% of the zinc sulfate; 0.15% the histidine, 0.2% of the vitamin B₁, 0.1% of the soybean oil, 0.1% of the adenine, 0.5% of the notoginseng power, and water provided for balance. The pH of the second culture medium in the seed tank is nature.

The third culture medium in the propagation tank comprises 3% of hot-pressed soybean meal powder, 3% of the glucose, 3% of sucrose; 0.15% of the potassium dihydrogen phosphate, 0.05% of the magnesium sulfate, 0.04% of the zinc sulfate; 0.1% the histidine, 0.15% of the vitamin B₁, 0.25% of the soybean oil, 0.03% of the adenine, 0.08% of the guanine, 0.1% of the uracil, 0.6% of the notoginseng powder, and water provided for balance.

The fourth culture medium in the fermentation tank comprises 3% of hot-pressed soybean meal powder, 2% of the glucose, 2% of sucrose; 0.3% of the potassium dihydrogen phosphate, 0.05% of the magnesium sulfate, 0.05% of the zinc sulfate; 0.3% the histidine, 0.2% of the vitamin B₁, 0.25% of the soybean oil, 0.1% of the adenine, 0.03% of the guanine, 0.1% of the uracil, 0.6% of the notoginseng powder, and water provided for balance.

The Cs-4 strains are fermented and cultivated under the same process conditions as in embodiment 1, and finally Cs-4 cordyceps fungus powder is obtained. content of the active ingredients in the cordyceps fungus powder prepared in the embodiment is slightly higher than that in the Embodiment 2, which is the result of the interaction between the guanine and the adenine.

### Embodiment 4

The slant culture medium comprises 4% of the glucose, 0.4% of the peptone, 0.8% of the bran, 0.3% of the potassium dihydrogen phosphate, 0.05% of the magnesium sulfate, 0.3% of the histidine, 4% of the agar, and the water provide for balance. The pH of the slant culture medium is nature. Then, the inoculation spatula is applied to pick up the Cs-4 strains of 1 cm² that has been stored, and the Cs-4 strains are evenly spread on the slant culture medium and cultivated for 9 days at a temperature of 20° C. and a humidity of 90%.

The first culture medium in the shake flask comprise 5% of the glucose, 0.4% of the peptone, 0.6% of the bran, 0.3% of the potassium dihydrogen phosphate, 0.05% of the magnesium sulfate, 0.05% of zinc sulfate, 0.3% of the histidine, 0.1% of the vitamin B₁, and the water provided for balance. The pH of the first culture medium in the shake flask is nature. Under aseptic conditions, the inoculation spatula is configured to take 1 cm² of the vigorously growing Cs-4 strains on the slant culture medium into the first culture medium in the shake flask. The shake flask is placed on the shaking table and the Cs-4 strains are shaken and cultivated at 20° C. for 9 days. The shaking frequency of the shaking table is 140 times/min. During an entire cultivation process in the shake flask, red light is used for irradiation. After the cultivation process in the shake flask is completed, the Cs-4 strains cultivated in each shake flask are collected into an inoculation steel cylinder

The second culture medium in the seed tank comprises 4% of hot-pressed soybean meal powder, 4% of the glucose, 4% of sucrose; 0.4% of the potassium dihydrogen phosphate, 0.1% of the magnesium sulfate, 0.1% of the zinc sulfate; 0.2% the histidine, 0.3% of the vitamin B₁, 0.2% of the soybean oil, 0.2% of the adenine, 0.6% of notoginseng powder, and water provided for balance. The pH of the second culture medium in the seed tank is nature. Under aseptic conditions, 5% by weight of the Cs-4 strains cultivated in the shake flask is inoculated into the second culture medium in the seed tank by pressure-difference method. The strains in the seed tank are cultivated at a constant temperature of 19° C. Pressure of the seed tank is 0.05 MPa, and ventilation volume of the seed tank is 1:1.8 v/v·min. The Cs-4 strains in the seed tank are statically cultivated for 5 days.

The third culture medium in the propagation tank comprises 4% of hot-pressed soybean meal powder, 4% of the glucose, 4% of sucrose; 0.2% of the potassium dihydrogen phosphate, 0.06% of the magnesium sulfate, 0.06% of the zinc sulfate; 0.2% the histidine, 0.3% of the vitamin B₁, 0.3% of the soybean oil, 0.1% of the adenine, 0.03% of the guanine, 0.2% of the uracil, 0.7% of the notoginseng powder, and water provided for balance. The pH of the third culture medium in the propagation tank is nature. Under aseptic conditions, 12% by weight of the Cs-4 strains cultivated in the seed tank is inoculated into the third culture medium in the propagation tank by the pressure-difference method. The Cs-4 strains in the propagation tank are cultivated at a constant temperature of 19° C. Pressure of the propagation tank is 0.05 MPa, and ventilation volume of the seed tank is 1:0.4 v/v·min. The Cs-4 strains in the propagation tank are statically cultivated for 6 days.

The fourth culture medium in the fermentation tank comprises 4% of hot-pressed soybean meal powder, 1% of the glucose, 1% of sucrose: 0.2% of the potassium dihydrogen phosphate, 0.06% of the magnesium sulfate, 0.06% of the zinc sulfate; 0.2% the histidine, 0.3% of the vitamin B₁, 0.3% of the soybean oil, 0.12% of the adenine, 0.08% of the guanine, 0.2% of the uracil, 1% of the notoginseng powder, and water provided for balance. Under aseptic conditions, 15% by weight of the Cs-4 strains cultivated in the propagation tank is inoculated into the fourth culture medium in the fermentation tank by the pressure-difference method. The Cs-4 strains in the fermentation tank are cultivated at a constant temperature of 19° C. Pressure of the fermentation tank is 0.05 MPa, and ventilation volume of the fermentation tank is 1:0.9 v/v·min. The Cs-4 strains in the fermentation tank are stirred and cultivated for 5 days at a rotating speed of 60 rpm. After cultivation in the fermentation tank is started, samples of the fermentation broth are taken every 24 hours to determine the pH, reducing sugars, and amino nitrogen. When pH<7, the reducing sugar<1.5%, the amino nitrogen<0.4 mg/mL, and bacteria concentration≥14%, the cultivation in the fermentation tank ends. Then the fermentation tank is opened to separate the fermentation liquid from solid and liquid, and the solid is dried and crushed to obtain Cs-4 cordyceps fungus powder.

The content of the active ingredients of the Cs-4 cordyceps fungus powder obtained in the embodiment is similar to the content of the active ingredients of the Cs-4 cordyceps fungus powder obtained in the embodiment 3.

### Embodiment 5

The only difference between this embodiment and the Embodiment 4 is that the Cs-4 strains to be cultivated in the shake flask is of the shake flask medium is the fermentation broth after the fermentation process of the Embodiment 4, and 1% of the fermentation broth is inoculated by weight. The Cs-4 strains are fermented and cultivated according to the same process conditions as in the Embodiment 4, and finally Cs-4 cordyceps fungus powder is obtained. The content of nucleosides, ergostero, ginsenoside, and notoginsenoside in the Cs-4 cordyceps fungus powder is higher than that of the Cs-4 cordyceps fungus powder obtained in the Embodiment 4, because the stains in the embodiment has a stronger ability to biotransform the purines, the pyrimidine, and the notoginseng powder.

### Embodiment 6

The components of each culture medium in the embodiment is same as that of Embodiment 4, the difference is that the Cs-4 strains to be cultivated in the shake flask is of the shake flask medium is the fermentation broth after the fermentation process of the Embodiment 4, and 2% of the fermentation broth is inoculated by weight. The shake flask is placed on the shaking table and the Cs-4 strains are shaken and cultivated at 18° C. for 8 days. The shaking frequency of the shaking table is 145 times/min. During an entire cultivation process in the shake flask, red light is used for irradiation.

The Cs-4 strains in the seed tank are cultivated at a constant temperature of 13° C. Pressure of the seed tank is 0.03 MPa, and ventilation volume of the seed tank is 1:1.0 v/v·min. The Cs-4 strains in the seed tank are statically cultivated for 4 days.

The Cs-4 strains in the propagation tank are cultivated at a constant temperature of 13° C. Pressure of the propagation tank is 0.03 MPa, and ventilation volume of the seed tank is 1.0.2 v/v·min. The Cs-4 strains in the propagation tank are statically cultivated for 5 days.

The Cs-4 strains in the fermentation tank are cultivated at a constant temperature of 13° C. Pressure of the fermentation tank is 0.03 MPa, and ventilation volume of the fermentation tank is 1:0.65 v/v·min. The Cs-4 strains in the fermentation tank are stirred and cultivated for 5 days at a rotating speed of 50 rpm.

After cultivation in the fermentation tank is completed, the fermentation tank is opened to obtain the Cs-4 cordyceps fungus powder. The content of the active ingredients of the Cs-4 cordyceps fungus powder obtained in the embodiment is similar to the content of the active ingredients of the Cs-4 cordyceps fungus powder obtained in the embodiment 5, which noted that the process parameter conditions of this embodiment are suitable.

### Control Embodiment 1

The difference between this control embodiment and the Embodiment 1 is that the second culture medium in the seed tank does not comprise the adenine, the third culture medium in the propagation tank does not comprise the adenine and the uracil, and the fourth culture medium in the fermentation tank does not comprise the adenine and the uracil. The Cs-4 strains are fermented and cultivated under the same process conditions as in the Embodiment 1, and finally the Cs-4 cordyceps fungus powder is obtained. The content of the nucleosides and the ergosterol in the Cs-4 cordyceps fungus powder prepared in this control embodiment is lower than that in the Embodiment 1.

### Control Embodiment 2

In the control embodiment, the notoginseng powder is added to the liquid culture mediums.

The difference between this control embodiment and the Embodiment 2 is that the second culture medium in the seed tank does not comprise the adenine, the third culture medium in the propagation tank does not comprise the adenine and the uracil, and the fourth culture medium in the fermentation tank does not comprise the adenine and the uracil. The Cs-4 strains are fermented and cultivated under the same process conditions as in the Embodiment 1, and finally the Cs-4 cordyceps fungus powder is obtained. The content of the nucleoside, the ergosterol, the ginsenoside, and the notoginsenoside in the Cs-4 cordyceps fungus powder prepared in this control embodiment is lower than that in the Embodiment 2, The low content of the ginsenoside and the notoginsenoside indicates that purines and the pyrimidine are able to promote the use of the notoginseng powder by the Cs-4 strains. Compared with the cordyceps fungus powder of the Control embodiment 1, the content of ergosterol in the cordyceps fungus powder obtained in this control embodiment is increased, indicating that the addition of the notoginseng powder is able to increase the content of ergosterol.

### Control Embodiment 3

The only difference between this control embodiment and the Embodiment 3 is that the Cs-4 strains in the first culture medium in the shake flask is cultivated under dark conditions, and the content of the active ingredients in the prepared Cs-4 cordyceps fungus powder is low, showing that the Cs-4 strains in the first culture medium in the shake flask need to be cultivated under light conditions.

### Control Embodiment 4

The only difference between this control embodiment and the control embodiment 3 is that the Cs-4 strains in the first culture medium in the shake flask is cultivated under natural light conditions, and the content of the active ingredients in the prepared Cs-4 cordyceps fungus powder is higher than that of the control embodiment 3 and lower than that of the Embodiment 3, showing that the red light is more conducive to the growth of the Cs-4 strains in the first culture medium in the shake flask.

### Control Embodiment 5

The difference between this control embodiment and the Embodiment 3 is that the Cs-4 strains in the shake flask is placed on the shaking table and the Cs-4 strains are shaken and cultivated at 25° C. for 8 days. The shaking frequency of the shaking table is 120 times/min. The Cs-4 strains in the seed tank are cultivated at a constant temperature of 20° C. Pressure of the seed tank is 0.03 MPa, and ventilation volume of the seed tank is 1:0.4 v/v·min. The Cs-4 strains in the seed tank are statically cultivated for 4 days. The Cs-4 strains in the propagation tank are cultivated at a constant temperature of 20° C. Pressure of the propagation tank is 0.03 MPa, and ventilation volume of the seed tank is 1:0.2 v/v·min. The Cs-4 strains in the propagation tank are statically cultivated for 5 days. The Cs-4 strains in the fermentation tank are cultivated at a constant temperature of 20° C. Pressure of the fermentation tank is 0.03 MPa, and ventilation volume of the fermentation tank is 1:0.65 v/v·min. The Cs-4 strains in the fermentation tank are statically cultivated for 5 days. After cultivation in the fermentation tank is completed, the fermentation tank is opened to obtain the Cs-4 cordyceps fungus powder. The content of the active ingredients of the Cs-4 cordyceps fungus powder obtained in the control embodiment is low, the main reason is that the culture conditions are not suitable, especially the Cs-4 strains in the fermentation tank cannot be cultivated statically.

### Embodiment 7

Samples are taken from the Cs-4 cordyceps fungus powder prepared in the Embodiments 1-6 and the Control Embodiments 1-5, to determine the contents of the nucleosides (adenosine, guanosine and uridine), the ergosterol, the ginsenoside, and the notoginsenoside. Test results are shown in Table 1. According to the test results, it is clearly that the Cs-4 cordyceps fungus powder produced by the fermentation production process of the Cs-4 strains provided by the present disclosure has high medicinal value.

TABLE 1
the contents of the nucleosides (adenosine, guanosine and uridine),
the ergosterol, the ginsenoside, and the notoginsenoside)

| | | | Saponin (mg/100 mg) |
|---|---|---|---|
| | | | (ginsenosideRg1, |
| | Nucleosides | Ergosterol | ginsenosideRb1, |
| | (mg/100 mg) | (mg/100 mg) | notoginsenoside R1) |
| Embodiment 1 | 0.58 | 0.51 | Not detected |
| Embodiment 2 | 0.60 | 0.72 | 6.9 |
| Embodiment 3 | 0.69 | 0.77 | 7.2 |
| Embodiment 4 | 0.68 | 0.76 | 7.4 |
| Embodiment 5 | 0.73 | 0.80 | 7.8 |
| Embodiment 6 | 0.72 | 0.81 | 8.0 |
| Control | 0.31 | 0.40 | Not detected |
| Embodiment 1 | | | |
| Control | 0.33 | 0.52 | 5.5 |
| Embodiment 2 | | | |
| Control | 0.29 | 0.43 | 4.8 |
| Embodiment 3 | | | |
| Control | 0.47 | 0.48 | 5.1 |
| Embodiment 4 | | | |
| Control | 0.44 | 0.42 | 4.6 |
| Embodiment 5 | | | |

The above are only optional specific embodiments of the present disclosure, but the protection scope of the present disclosure is not limited thereto. Those skilled in the art can easily think of substitution or changes within the technical scope disclosed by the present disclosure, which shall be fall within the protection scope of the present disclosure.

What is claimed is:

1. A fermentation production process of paecilomyces hepialis Cs-4, comprising following steps:
step 1: inoculating Cs-4 strains on a slant culture medium and cultivating the Cs-4 strains for 7-9 days at a constant temperature in a range of 15-20° C.; wherein the slant culture medium comprises histidine;
step 2. inoculating the Cs-4 strains cultivated in step 1 into a first culture medium in a shake flask under aseptic conditions; placing the shake flask on a shaking table and cultivating the Cs-4 strains in the shake flask under light conditions for 7-9 days at a constant temperature in a range of 15-20° C.; wherein the first culture medium in the shake flask comprises the histidine and vitamin B₁;
step 3. inoculating the Cs-4 strains cultivated in step 2 in an amount ranging from 3-5% by weight into a second culture medium in a seed tank under aseptic conditions; wherein the second culture medium in the seed tank comprises purine;
step 4. inoculating the Cs-4 strains cultivated in step 3 in an amount ranging from 9-12% by weight into a third culture medium in a propagation tank under aseptic conditions; wherein the third culture medium in the propagation tank comprises purine and pyrimidine;
step 5. inoculating the Cs-4 strains cultivated in step 4 in an amount ranging from 5-20% by weight into a fourth culture medium in a fermentation tank and obtaining fermentation broth after cultivation in the fermentation tank is finished; wherein the fourth culture medium in the fermentation tank comprises purine and the pyrimidine;
or, the Cs-4 strains obtained in step 5 are obtained by:
inoculating the fermentation broth obtained in step 5 in an amount ranging from 1-2% by weight into the first culture medium in the shake flask, and then cultivating the fermentation broth under conditions of step S2, and then repeating steps 3-5 to ferment and cultivate the Cs-4 strains.

2. The fermentation production process according to claim 1, wherein the purine is selected from one or two of adenine and guanine; the pyrimidine is uracil.

3. The fermentation production process according to claim 1, wherein based on weight-to-volume ratio (g/mL), the slant culture medium comprises glucose in an amount ranging from 2-4%, peptone in an amount ranging from 0.2-0.4%, bran in an amount ranging from 0.4-0.8%, potassium dihydrogen phosphate in an amount ranging from 0.1-0.3%, magnesium sulfate in an amount ranging from 0.03-0.05%, the histidine in an amount ranging from 0.1-0.3%, agar in an amount ranging from 2-4%, and water provide for balance;
wherein the first culture medium in the shake flask comprise the glucose in an amount ranging from 1-5%, the peptone in an amount ranging from 0.2-0.4%, the bran in an amount ranging from 0.4-0.6%, the potassium dihydrogen phosphate in an amount ranging from 0.1-0.3%, the magnesium sulfate in an amount ranging from 0.03-0.05%, zinc sulfate in an amount ranging from 0.03-0.05%, the histidine in an amount ranging from 0.1-0.3%, the vitamin B₁ in an amount ranging from 0.05-0.1%, and the water provided for balance.

4. The fermentation production process according to claim 1, wherein the second culture medium in the seed tank, the third culture medium in the propagation tank, and the fourth culture medium in the fermentation tank comprise notoginseng powder.

5. The fermentation production process according to claim 1, wherein based on weight-to-volume ratio (g/mL), the second culture medium in the seed tank comprises hot-pressed soybean meal powder in an mount ranging from 2-4%, glucose in an mount ranging from 2-4%, sucrose in an mount ranging from 2-4%, potassium dihydrogen phosphate in an mount ranging from 0.2-0.4%, magnesium sulfate in an mount ranging from 0.05-0.1%, zinc sulfate in an mount ranging from 0.05-0.1%, the histidine in an mount ranging from 0.1-0.2%, the vitamin B₁ in an mount ranging from 0.02-0.3%, soybean oil in an mount ranging from 0.1-0.2%, adenine in an mount ranging from 0.05-0.2% notoginseng powder in an mount ranging from 0.4-0.6%, and water provided balance.

6. The fermentation production process according to claim 1, wherein based on weight-to-volume ratio (g/mL), the third culture medium in the propagation tank comprises hot-pressed soybean meal in an mount ranging from 2-4%, glucose in an mount ranging from 2-4%, sucrose in an mount ranging from 2-4%, potassium dihydrogen phosphate in an mount ranging from 0.1-0.2%, magnesium sulfate in an mount ranging from 0.04-0.06%, zinc sulfate in an mount ranging from 0.03-0.05%, the histidine in an mount ranging from 0.1-0.2%, the vitamin B₁ in an mount ranging from 0.1-0.2%, soybean oil in an mount ranging from 0.2-0.3%, adenine in an mount ranging from 0.03-0.1%, guanine in an mount ranging from 0.03-0.08%, uracil in an mount ranging from 0.05-0.2%, notoginseng powder in an mount ranging from 0.5-0.7%, and water provided for balance.

7. The fermentation production process according to claim 1, wherein based on weight-to-volume ratio (g/mL), the fourth culture medium in the fermentation tank comprises hot-pressed soybean meal in an mount ranging from 3-4%, glucose in an mount ranging from 1-2%, sucrose in an mount ranging from 1-2%, potassium dihydrogen phosphate in an mount ranging from 0.2-0.3%, magnesium sulfate in an mount ranging from 0.04-0.06%, zinc sulfate in an mount ranging from 0.04-0.06%, the histidine in an mount ranging from 0.2-0.3%, the vitamin B₁ in an mount ranging from 0.2-0.3%, soybean oil in an mount ranging from 0.2-0.3%, adenine in an mount ranging from 0.05-0.12%, guanine in an mount ranging from 0.03-0.08%, uracil in an mount ranging from 0.05-0.2%, notoginseng powder in an mount ranging from 0.5-1.0%, and water provided for balance.

8. The fermentation production process according to claim 7, wherein the shaking table is a reciprocating shaking table; a shaking frequency of the shaking table is in a range of 140±5 times/min; light used in the light condition is red light; the Cs-4 strains in the seed tank are cultivated at a constant temperature in a range of 16±3° C.; pressure of the seed tank is in a range of 0.02-0.05 MPa, ventilation volume of the seed tank is 1:0.5-1.8 v/v·min; the Cs-4 strains in the seed tank are statically cultivated for 4-5 days.

9. The fermentation production process according to claim 8, wherein the Cs-4 strains in the propagation tank are cultivated at a constant temperature in a range of 16±3° C.; pressure of the propagation tank is in a range of 0.02-0.05 MPa, ventilation volume of the propagation tank is 1:0.15-0.4 v/v·min; the Cs-4 strains in the propagation tank are statically cultivated for 5-6 days.

10. The fermentation production process according to claim 9, wherein the Cs-4 strains in the fermentation tank are cultivated at a constant temperature in a range of 16±3° C.; pressure of the fermentation tank is in a range of 0.02-0.05 MPa, ventilation volume of the fermentation tank is 1:0.65-0.9 v/v·min; the Cs-4 strains in the fermentation tank are stirred and cultivated for 4-5 days at a rotating speed ranging from 40-60 rpm.

11. The fermentation production process according to claim 9, wherein ventilation volume of the fermentation tank is 1:0.75 v/v·min.
